(19) United States
(12) Patent Application Publication
(10) Pub. No.: US 2025/0295345 A1
(43) Pub. Date: Sep. 25, 2025

(54) HAND-HELD MEASUREMENT APPARATUS, SYSTEMS, AND METHODS

(71) Applicant: The Trustees of Indiana University, Bloomington, IN (US)

(72) Inventor: Sergiu Iulian Abramovici, Carmel, IN (US)

(21) Appl. No.: 19/083,739

(22) Filed: Mar. 19, 2025

Related U.S. Application Data
(60) Provisional application No. 63/567,578, filed on Mar. 20, 2024.

(51) Int. Cl. A61B 5/291; A61B 5/26; A61B 5/294; A61B 5/377

(52) U.S. Cl. CPC A61B 5/291; A61B 5/26; A61B 5/294; A61B 5/377

(57) ABSTRACT

Devices, methods, kits, and systems for recording electrophysiological signals, such as an electroencephalogram, from a subject. A hand-held device for electrophysiological signal recording from a subject may include: a flexible substrate having a plurality of electrodes, e.g., a 5×5 matrix of electrodes, mounted onto an external surface of the substrate and circuitry configured to acquire and process electrophysiological signals recorded from a subject by the electrodes and for communicating the recorded electrophysiological signals to a remote device.

### CROSS-REFERENCE TO RELATED APPLICATION

This application claims priority to U.S. Provisional Patent App. No. 63/567,578, filed Mar. 20, 2024, the entire content of which is incorporated herein by reference.

### BACKGROUND

The present disclosure relates to a hand-held device for acquiring and/or monitoring data from a subject, and, more particularly, but not exclusively, to a hand-held device and interface configured for obtaining and processing electrophysiological data, such as electroencephalogram (EEG).

### SUMMARY

According to an aspect, the present description provides a hand-held device for electrophysiology data recording from a subject. More particularly, the present description provides a hand-held device for electrophysiological signal recording from a subject, comprising: a flexible substrate having a plurality of electrodes mounted onto an external surface of the substrate; and circuitry configured to acquire and process electrophysiological signals recorded from a subject by the electrodes and for communicating the recorded electrophysiological signals to a remote device.

In some embodiments, the flexible substrate comprises an electrically insulating material.

In some embodiments, the flexible substrate has a reverse surface, with an electrically insulating lining covering the reverse surface of the flexible substrate. In some embodiments, the flexible substrate forms a 3-dimensional shape sphere, hemisphere, spheroid, polyhedron, cylinder, torus, toroid, and hyperboloid.

In some embodiments, the circuitry includes an analog to digital converter, an amplifier, and a communication module.

In some embodiments, each of the two or more electrodes are at a fixed distance on the external surface of the substrate.

In some embodiments, the electrodes may be arranged in an x×y matrix, wherein x and y are, independently, integers from 1 to 10. In any embodiment, the matrix may be a 5×5 matrix.

In some embodiments, the electrodes are dry electrodes, each electrode comprising a plurality of pins long enough for contacting the subject's scalp through hair.

In some embodiments, the pins protrude at least 2 mm away from the external surface of the substrate.

In some embodiments, the circuitry comprises a controller programmed to control recording by the electrodes based on input received from the remote device.

In some embodiments, the substrate comprises at least one spring positioned between the electrode and the external surface of the substrate which provides resistance in response to pressure applied onto the electrode.

In some embodiments, the device comprises one or more optical markers positioned on the external surface, the optical markers detectable by the remote device.

In any embodiment, the subject may be a human patient.

In another aspect, the present disclosure provides a system comprising: a device for example as referred to herein, and a remote device including a user interface, the user interface configured for receiving input data and for generating a subject condition related indication based on the input data and on the electrophysiological signals recorded by the electrodes.

In some embodiments, the circuitry is programmed to communicate with the remote device and the remote device is programmed to control recording by the electrodes.

In some embodiments, the input data includes a current position of the electrodes with respect to the subject's head.

In some embodiments, the input data includes one or more of: vital signs, symptoms exhibited by the subject; an assumed medical condition, an associated event which the subject was involved in, and the subject's medical and surgical history.

In some embodiments, the user interface comprises a screen for presenting operating instructions to a user relating to the position of the electrodes with respect to the subject's head.

In some embodiments, the remote device comprises a smart phone or a tablet computer.

In some embodiments, the remote device comprises: an imager; and circuitry configured to identify a position of the electrodes with respect to the subject's anatomy from images acquired by the imager.

In some embodiments, the user interface is configured as augmented reality glasses to be worn by a user.

In some embodiments, the glasses are configured to present to a user one or more of: instructions for positioning the hand-held device on the subject; a duration for measuring; currently recorded data; subject condition related indications.

In some embodiments, one or both of the hand-held device and the remote device are configured to transfer and/or receive data to and/or from a medical center database.

In some embodiments, the data includes the subject condition related indication generated by the system.

In some embodiments, the data includes follow-up instructions generated based on the subject related condition indication.

In some embodiments, follow up instructions include recommendations for imaging the subject and/or for the type of imaging modality required and/or the anatomical region deemed relevant for imaging.

In some embodiments, the remote device is configured for generating the subject related condition indication within 3-6 minutes of recording the electrophysiological signals.

In any embodiment, the subject may be a human patient.

In still another aspect, the present disclosure provides a kit comprising: a set of two of the hand-held device as described herein, wherein at least one of the devices further comprises a stimulation module for applying an electrical, visual and/or audible stimulation to the subject. In any embodiment of the kit, the subject may be a human patient.

According to yet another aspect, the present description provides a method for recording electrophysiological signal of a subject using a hand-held device comprising a plurality of electrodes, comprising: a user holding the hand-held device in the user's hand; contacting the plurality of electrodes with the surface of the subject's body; and recording electrophysiological signals. In any embodiment, the electrophysiological signal may be any of electroencephalography (EEG), electrocardiogram (ECG) data; nerve conduction velocity (NCV) data; visual evoked potentials (VEP); brainstem auditory evoked potentials (BAEP); somatosensory evoked potentials (SSEP); or a combination thereof.

In some embodiments, the method comprises obtaining an automatically generated indication related to a condition of the subject, the indication at least partially based on the recorded EEG signals.

In some embodiments, recording is for a time period of between 5-30 seconds for each recording position or epoch.

In some embodiments, the method comprises adjusting a position of the device relative to the subject's head to reposition at least some of the plurality of electrodes on the scalp.

In some embodiments, the hand-held device comprises or is in communication with a user interface and the placing and/or the adjusting are according to instructions provided by the user interface.

In some embodiments, recording comprises setting the electrodes in a bipolar montage or in a weighted average montage.

In some embodiments, in the bipolar montage, one of the plurality of electrodes is used as a reference and ground and in the weighted average montage one of the plurality of electrodes is used as common average. In some embodiments, a Laplacian montage may be calculated.

In some embodiments, the method comprises repeating the placing and recording steps at a plurality of different positions relative to the subject's scalp.

In some embodiments, the indication related to a condition of the subject is generated by determining patterns in the recorded EEG signals using feature extraction techniques and comparing the patterns to pre-established EEG patterns known to be associated with physiological and/or pathophysiological conditions.

In some embodiments, the indication related to a condition of the subject is from the group of: cerebrovascular disease, central nervous system tumor, encephalitis, head trauma, epilepsy, encephalopathy, headache, back pain, spinal cord pathology, multiple sclerosis, other myelopathy, limb weakness/numbness, visual disturbance, unilateral hearing loss.

In any embodiment, the subject may be a human patient.

In still another aspect, the present disclosure provides a method for stimulating a subject and detecting a response to the stimulation using a hand-held device comprising a plurality of electrodes, comprising: applying an electrical stimulation to an affected limb; and using the hand-held device, recording a response to the stimulation via the plurality of electrodes.

In some embodiments, the electrodes comprise EEG electrodes.

In some embodiments, the response comprises an evoked potential of the nervous system.

In some embodiments, applying is via a stimulating device comprising at least an anode, cathode and grounding elements.

In some embodiments, recording comprises positioning the hand-held device on the subject's head such that the electrodes are in contact with the scalp.

In some embodiments, recording comprises positioning the hand-held device on any anatomical area such that the electrodes contact the skin.

In some embodiments, an electrical stimulation is applied to a subject's upper limb and/or lower limb.

In some embodiments, recording is performed at one or more locations along a neural path leading to the stimulated anatomical area.

In any embodiment, the subject may be a human patient.

In a further aspect, the present disclosure provides a hand-held device for recording of electrophysiological data from a subject, comprising: a matrix comprising a flexible substrate, at least three electrodes, each electrode mounted on an external surface of the flexible substrate; and circuitry for activating the electrodes to record electrophysiology related signals from a subject and for communicating the recorded signals to a remote device.

In some embodiments, the electrophysiology data comprises at least one of: Electroencephalography (EEG), Electrocardiogram (ECG) data; Nerve Conduction Velocity (NCV) data; Visual Evoked Potentials (VEP); Brainstem Auditory Evoked Potentials (BAEP); Somatosensory Evoked Potentials (SSEP).

In an additional aspect, the present disclosure provides a hand-held device for EEG recording of a subject, comprising: a matrix comprising a flexible substrate, the substrate having an external surface with a plurality of electrodes mounted thereon, each electrode shaped for contacting the subject's scalp for recording EEG signals; and circuitry configured to acquire and process signals recorded from a subject by the electrodes and for communicating the recorded signals to a remote device.

In any embodiment, the subject may be a human patient.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a flowchart of a general method for recording electroencephalogram (EEG) signals using a hand-held device, according to some embodiments of methods of the present disclosure;

FIG. 2 is a diagram of a basic system for recording EEG signals using a hand-held device, according to some embodiments according to the present disclosure;

FIGS. 3A-3B schematically illustrate examples of positions and movements of a hand-held device comprising electrodes on a subject's head, according to some embodiments, wherein FIG. 3A shows five positions on the back of a human subject's head from which readings may be taken and averaged, and FIG. 3B shows five positions on the side of a human subject's head from which readings may be taken and averaged;

FIG. 4 is a flowchart of a method of generating a subject condition indication based on data acquired by a hand-held device, according to some embodiments of methods of the present disclosure;

FIGS. 5A-5B are diagrams illustrating operation of systems according to embodiments of the present disclosure, wherein FIG. 5A shows an embodiment without a subject with wearable electrostimulators and FIG. 5B shows an embodiment with a subject wearing electrostimulator bracelets on the subject's right wrist and wright ankle;

FIGS. 6A-6B are exemplary interactive device interfaces, wherein FIG. 6A is an exemplary user interface (UI) display mapping diagram of human patient neurophysiological symptoms and FIG. 6B is an exemplary UI display whereby a user may input additional patient symptoms;

FIG. 7 is additional interactive device interfaces, wherein the left side of the drawing shows an exemplary UI indicating characteristics of an electrophysiological reading, and which then relates to possible medical diagnoses, as shown in an exemplary UI on the right side of the drawing';

FIGS. 8A-8E are non-limiting exemplary electrophysiological reading applications for which the apparatus and systems of the present disclosure may be used, including electroencephalogram (FIG. 8A), brainstem auditory evoked potential (FIG. 8B), electrocardiogram (FIG. 8C), nerve conduction velocity (FIG. 8D), and visual evoked potential/response (FIG. 8E);

FIG. 9 is an exemplary illustration of an operation of apparatus and systems of the present disclosure, wherein a human subject is fitted with wrist and ankle electrodes for generating somatosensory evoked potentials (SSEP), and wherein the handheld device is moved to locations on the subject's body, including chest, neck, and locations on the subject's head;

FIGS. 10A-10B are exemplary diagrams of a geometric arrangement of electrodes on surface of the device of the present disclosure, and without (FIG. 10A) and with (FIG. 10B) near-infrared spectroscopy (NIRS) sensors;

FIG. 11 is an cartoon illustration of a user's hand grasping an embodiment of the hand-held device of the present disclosure, and indicating placement of the electrodes over a human subject's brain;

FIGS. 12A-12C are illustrations of expected electrophysiological readings in the case of different injury conditions, wherein FIG. 12A shows four exemplary readings for a patient having a subdural hematoma (SDH) on a patient's right hemisphere, FIG. 12B shows four exemplary readings for a patient having a large vessel occlusion (LVO) on a patient's right hemisphere, and FIG. 12C shows four exemplary readings for a patient having a skull fracture and breach on a patient's left hemisphere;

FIGS. 13A-13B are exemplary illustrations of blood vessel obstruction in a section of brain tissue, illustrating the placement and effect of an obstruction in the case of LVO (FIG. 13A), with the gray shading indicating area of affected tissue, as compared with small-vessel stroke (FIG. 13B); and

FIGS. 14A-14C exemplary use and readouts for devices and systems of the present disclosure in different injury scenarios, including brain injury (FIG. 14A), cervical (i.e., neck) injury (FIG. 14B), and lower spinal injury (FIG. 14C).

### DETAILED DESCRIPTION

The present disclosure, in some embodiments thereof, relates to a hand-held device for monitoring subject data, and, more particularly, but not exclusively, to a hand-held device and interface configured for obtaining and processing electrophysiological data, such as EEG data.

According to an aspect, the present description provides a hand-held device for electrophysiology data recording from a subject. More particularly, the present description provides a hand-held device for EEG recording from a subject, comprising: a flexible substrate having a plurality of electrodes mounted onto an external surface of the substrate; and circuitry configured to acquire and process EEG signals recorded from a subject by the electrodes and for communicating the recorded EEG signals to a remote device.

In some embodiments, the flexible substrate comprises an electrically insulating material.

In some embodiments, the flexible substrate has a reverse surface, with an electrically insulating lining covering the reverse surface of the flexible substrate. In some embodiments, the flexible substrate forms a 3-dimensional shape sphere, hemisphere, spheroid, hemispheroid, ellipsoid, polyhedron, cylinder, torus, toroid, and hyperboloid.

In some embodiments, the circuitry includes an analog to digital converter, an amplifier, and a communication module.

In some embodiments, each of the two or more electrodes are at a substantially fixed distance on the external surface of the substrate. In some embodiments, each of the two or more electrodes are at a fixed distance on the external surface of the substrate. Advantageously, fixing the position of electrodes on the substrate allows for standard measurement and/or estimation of resistance, potentials across the distance of the gap between electrodes.

In some embodiments, the electrodes are dry electrodes, each electrode comprising a plurality of pins long enough for contacting the subject's scalp through hair.

In some embodiments, the pins protrude at least 2 mm away from the external surface of the substrate.

In some embodiments, the circuitry comprises a controller programmed to control recording by the electrodes based on input received from the remote device.

In some embodiments, the substrate comprises at least one spring positioned between the electrode and the external surface of the substrate which provides resistance in response to pressure applied onto the electrode.

In some embodiments, the device comprises one or more optical markers positioned on the external surface, the optical markers detectable by the remote device.

In any embodiment, the subject may be a human patient.

In another aspect, the present disclosure provides a system comprising: a device for example as referred to herein, and a remote device including a user interface, the user interface configured for receiving input data and for generating a subject condition related indication based on the input data and on the EEG signals recorded by the electrodes.

In some embodiments, the circuitry is programmed to communicate with the remote device and the remote device is programmed to control recording by the electrodes.

In some embodiments, the input data includes a current position of the electrodes with respect to the subject's head.

In some embodiments, the input data includes one or more of: vital signs, symptoms exhibited by the subject; an assumed medical condition, an associated event which the subject was involved in, and the subject's medical and surgical history.

In some embodiments, the user interface comprises a screen for presenting operating instructions to a user relating to the position of the electrodes with respect to the subject's head.

In some embodiments, the remote device comprises a smart phone or a tablet computer.

In some embodiments, the remote device comprises: an imager; and circuitry configured to identify a position of the electrodes with respect to the subject's anatomy from images acquired by the imager.

In some embodiments, the user interface is configured as augmented reality glasses to be worn by a user.

In some embodiments, the glasses are configured to present to a user one or more of: instructions for positioning the hand-held device on the subject; a duration for measuring; currently recorded data; subject condition related indications.

In some embodiments, one or both of the hand-held device and the remote device are configured to transfer and/or receive data to and/or from a medical center database.

In some embodiments, the data includes the subject condition related indication generated by the system.

In some embodiments, the data includes follow-up instructions generated based on the subject related condition indication.

In some embodiments, follow up instructions include recommendations for imaging the subject and/or for the type of imaging modality required and/or the anatomical region deemed relevant for imaging.

In some embodiments, the remote device is configured for generating the subject related condition indication within 3-6 minutes of recording the electrophysiological signals.

In any embodiment, the subject may be a human patient. In principle, the devices and systems of the present disclosure may be used in any organism having a nervous system. Accordingly, in any embodiment, the subject may be an animal. In any embodiment, the subject may be a vertebrate. In any embodiment, the subject may be a mammal. In any embodiment, the subject may be a cow, horse, pig, dog, or cat.

In still another aspect, the present disclosure provides a kit comprising: a set of at least one of the hand-held device as described herein, and one electric stimulation device for applying an electrical, visual and/or audible stimulation to the subject. In an embodiment, the kit may comprise a set of two of the hand-held devices as described herein, wherein at least one of the devices further comprises a stimulation module for applying an electrical, visual and/or audible stimulation to the subject. In any embodiment of the kit, the subject may be a human patient.

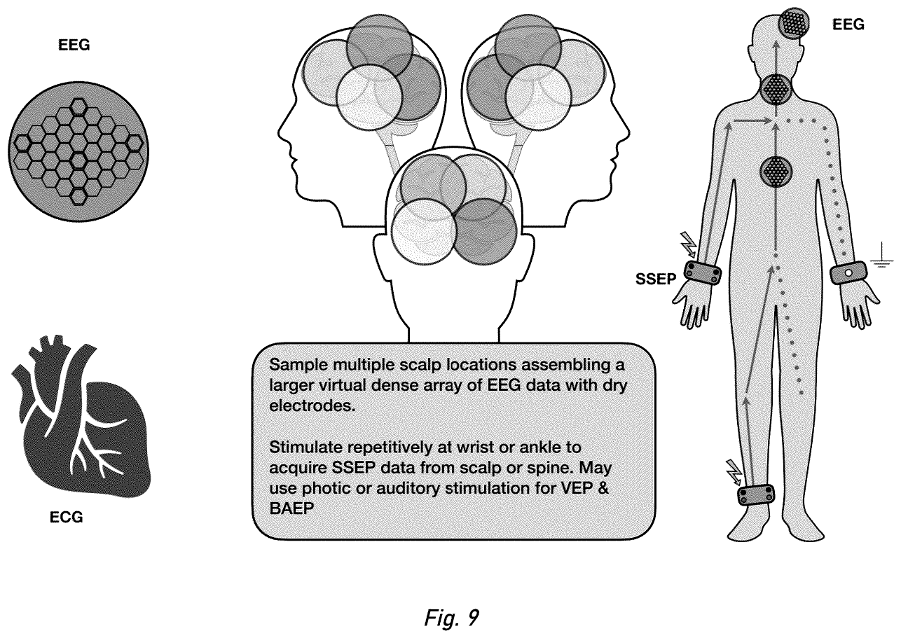

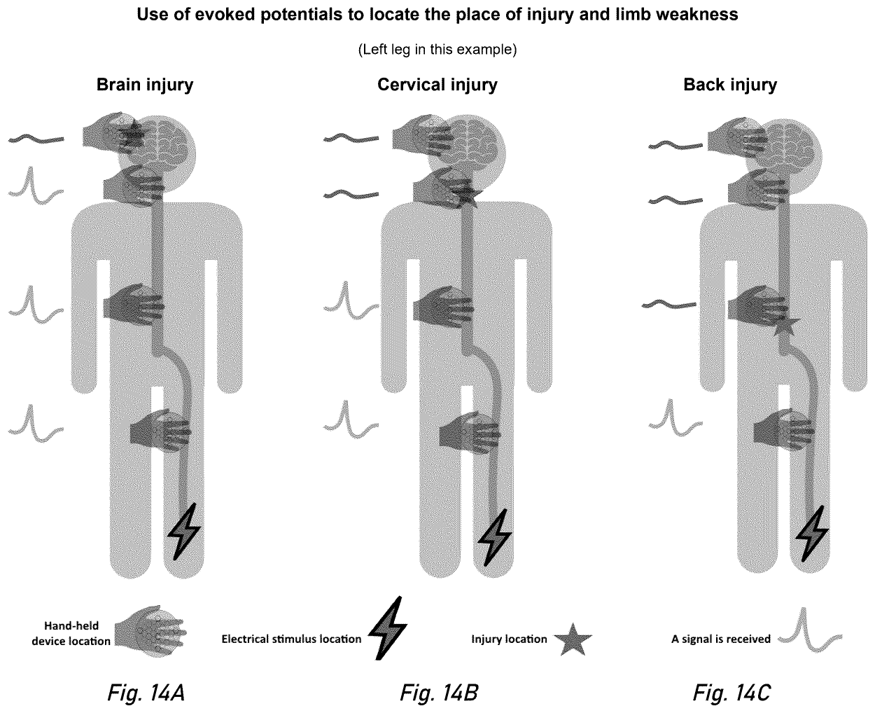

It is contemplated that, for example, an electric stimulation device, configured, for example as a cuff, may be placed on a subject's ankle, wrist, or devices may be placed on both ankle and wrist, and deliver mild electrical stimulus to the subject. (FIGS. 9, 14A-14C.) The subject may also be fitted with a grounding device, which may be configured as a cuff or bracelet. (FIG. 9.) Such stimulus may occur at a pre-programmed pattern, or upon an activation input by a user. The hand-held device of the present disclosure may then be moved by a user to different placements along the subject's body. For instance, electrical stimulus may be applied from a stimulation device to the subject's ankle. In a healthy patient without nerve injury, the electrical stimulus would be expected to deliver a signal from the ankle to the subject's spinal cord and up to the brain. Supposing, upon stimulus at the subject's ankle, that the hand-held device detects an electrical signal at the subject's lumbar vertebral area, but does not detect a signal at the subject's cervical vertebral area, the site of nerve injury may be located between these two sites. (FIG. 14C.)

According to yet another aspect, the present description provides a method for recording electrophysiological signals of a subject using a hand-held device comprising a plurality of electrodes, comprising: a user holding the hand-held device in the user's hand; contacting the plurality of electrodes with the surface of the subject's body; and recording electrophysiological signals. In any embodiment, the electrophysiological signal may be any of electroencephalography (EEG), electrocardiogram (ECG) data; nerve conduction velocity (NCV) data; visual evoked potentials (VEP); brainstem auditory evoked potentials (BAEP); somatosensory evoked potentials (SSEP); or a combination thereof. In any embodiment, the electrophysiological signals may be an EEG. In any embodiment, the method may comprise using a hand-held device comprising a plurality of electrodes, comprising: a user holding the hand-held device in the user's hand; contacting the plurality of electrodes with the subject's scalp; and recording EEG signals.

In some embodiments, the method may comprise obtaining an automatically generated indication related to a condition of the subject, the indication at least partially based on the recorded electrophysiological signals.

In some embodiments, recording is for a time period of between 5-30 seconds for each recording position or epoch.

In some embodiments, the method comprises adjusting a position of the device relative to the surface of the subject's body to reposition at least some of the plurality of electrodes on the surface of the body. In an embodiment, the method may comprise adjusting a position of the device relative to the subject's head to reposition at least some of the plurality of electrodes on the scalp.

In some embodiments, the hand-held device comprises or is in communication with a user interface and the placing and/or the adjusting are according to instructions provided by the user interface.

In some embodiments, recording comprises setting the electrodes in a bipolar montage or in a weighted average montage.

In some embodiments, in the bipolar montage, one of the plurality of electrodes is used as a reference and ground and in the weighted average montage one of the plurality of electrodes is used as common average.

In some embodiments, the method comprises repeating the placing and recording steps at a plurality of different positions relative to the subject's scalp.

In some embodiments, the indication related to a condition of the subject is generated by determining patterns in the recorded electrophysiological signals using feature extraction techniques and comparing the patterns to pre-established electrophysiological patterns known to be associated with physiological and/or pathophysiological conditions. In any embodiment, the electrophysiological signal may be an EEG.

In some embodiments, the indication related to a condition of the subject is from the group of: a cerebrovascular disease, central nervous system tumor, encephalitis, head trauma, skull fracture, epilepsy, encephalopathy, headache, back pain, spinal cord pathology, multiple sclerosis, other myelopathy, limb weakness/numbness, visual disturbance, unilateral hearing loss, or any combination thereof.

In any embodiment, the subject may be a human patient.

In still another aspect, the present disclosure provides a method for stimulating a subject and detecting a response to the stimulation using a hand-held device comprising a plurality of electrodes, comprising: applying an electrical stimulation to an affected limb; and using the hand-held device, recording a response to the stimulation via the plurality of electrodes.

In some embodiments, the electrodes comprise EEG electrodes.

In some embodiments, the response comprises an evoked potential of the nervous system.

In some embodiments, applying is via a stimulating device comprising at least an anode, cathode and grounding elements.

In some embodiments, recording comprises positioning the hand-held device on the subject's head such that the electrodes are in contact with the scalp.

In some embodiments, recording comprises positioning the hand-held device on any anatomical area such that the electrodes contact the skin.

In some embodiments, an electrical stimulation is applied to a subject's upper limb and/or lower limb.

In some embodiments, recording is performed at one or more locations along a neural path leading to the stimulated anatomical area.

In any embodiment, the subject may be a human patient.

In a further aspect, the present disclosure provides a hand-held device for recording of electrophysiological data from a subject, comprising: a matrix comprising a flexible substrate, at least three electrodes, each electrode mounted on an external surface of the flexible substrate; and circuitry for activating the electrodes to record electrophysiology related signals from a subject and for communicating the recorded signals to a remote device.

In some embodiments, the electrophysiology data comprises at least one of: Electroencephalography (EEG), Electrocardiogram (ECG) data; Nerve Conduction Velocity (NCV) data; Visual Evoked Potentials (VEP); Brainstem Auditory Evoked Potentials (BAEP); Somatosensory Evoked Potentials (SSEP).

In an additional aspect, the present disclosure provides a hand-held device for EEG recording of a subject, comprising: a matrix comprising a flexible substrate, the substrate having an external surface with a plurality of electrodes mounted thereon, each electrode shaped for contacting the subject's scalp for recording EEG signals; and circuitry configured to acquire and process signals recorded from a subject by the electrodes and for communicating the recorded signals to a remote device.

In any embodiment, the subject may be a human patient. In any embodiment, the subject may be an animal. In any embodiment, the subject may be a vertebrate. In any embodiment, the subject may be a mammal. In any embodiment, the subject may be a cow, horse, pig, dog, or cat.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

A broad aspect of some embodiments relates to recording data from a subject by placing a hand-held device comprising a plurality of electrodes on an anatomical area of the subject. In some embodiments, electrodes are placed at a selected spatial arrangement, and signals are recorded over a relatively short period of time ranging, for example, between 10-60 seconds. In some embodiments, recording is performed from a plurality of different spatial arrangements, and the user grasping the hand-held device adjusts the electrode positions by moving the device relative to the subject's body.

It will be appreciated that the devices, methods, systems, and kits of the present disclosure may have application in a variety of contexts and human and animal subjects, including, but not limited to, human, ape, monkey, dog, cat, horse, cow, pig, sheep, goat, elephant, and rodent.

In some embodiments, rapid collecting of data from the subject is enabled due to that no or only little setup is required, allowing the user to immediately place the hand-held device and initiate recording, optionally adjusting the electrode position in real time. For these reasons, hand-held devices and methods of use for example as described herein may be especially advantageous in emergency situations, pre-hospital settings, and/or other locations or situations in which a simple, dynamic assessment of the subject condition is required.

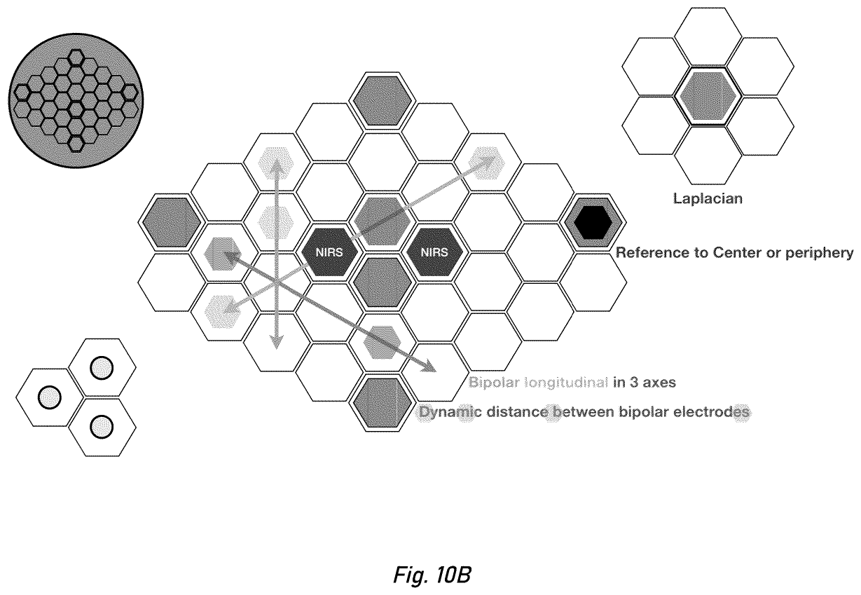

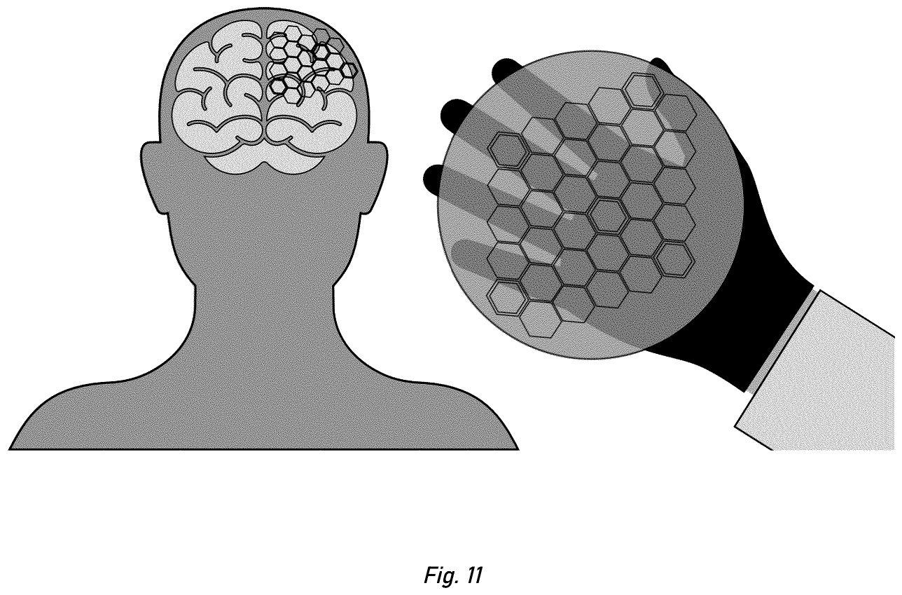

An aspect of some embodiments relates to a hand-held device suitable for non-invasive acquisition and monitoring electrophysiological signals of a subject, such as a subject's EEG. In some embodiments, the hand-held device is in the form of matrix made from a flexible substrate, formed into a substantially fixed 3-dimensional shape. The flexible substrate my comprise an elastic or semi-elastic; a stretched synthetic, natural, or mixed-fiber (i.e., synthetic and natural) textile; springs (e.g., pneumatic or coil); semi-rigid material; spongy material; woven or unwoven fabric material; or other suitable means of providing a flexible substrate in a substantially fixed 3-dimensional shape. The 3-dimensional shape may be, without limitation, sphere, hemisphere, bowl-shaped, spheroid, cylinder, cylindroid, cube, cuboid, square pyramid, conic, polyhedron, cylinder, torus, toroid, or hyperboloid. For instance, the hand-held device may comprise an array of electrodes mounted on the surface of a matrix made from an artificial sponge material having a baseline spheroid shape. A user may gently press the electrodes onto the surface of a subject's body, e.g., on the subject's scalp, and the sponge material will depress, thereby permitting the electrodes of the electrode array to conform to the contour of the subject's body, and remain in conductive contact with the surface. Upon removing the hand-held device from the surface of the subject's body, the device substrate returns to its baseline shape conformation. (FIG. 11.)

In some embodiments, a user holding the device places it on the subject's head such that the electrodes are in operable contact with the scalp. In some embodiments, various device positions (e.g., electrode positions) for recording during a plurality of epochs are selected based on the clinical context (symptoms exhibited by the subject, an event the subject was involved in [e.g., a human patient in a car accident], a known premorbid condition, etc.). Additionally, or alternatively, the device is used in a scanning mode, in which the user places the hand-held device at various spatial positions until detecting abnormal findings (for example, certain EEG patterns) which may be indicative of the subject's condition.

In an embodiment, the device comprises at least five electrodes. In an exemplary embodiment, the device comprises a x×y matrix of electrodes, wherein x is an integer from 1 to 10 and y is an integer from 1 to 10. In such embodiments, the product of x*y may be at least 5. In one embodiment, the device comprises a 5×5 matrix of electrodes. (FIG. 11.) The device may comprise at least 5, at least 10, at least 15, at least 20, at least 25, at least 30, or more electrodes. In one embodiment, the device comprises 30 electrodes.

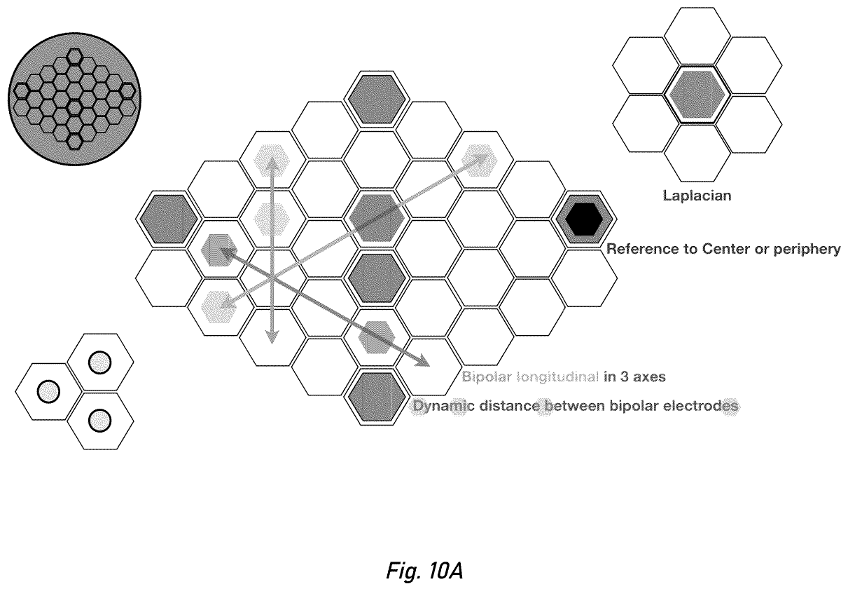

In an exemplary embodiment, using the 5×5 electrodes, different recording channels may be obtained. The user may position the matrix of electrodes at various arrangements with respect to each other to obtain a selected recording montage, such as a longitudinal (sagittal) or transverse (coronal) bipolar recording montages, or a weighted average recording montage. (FIG. 10A-10B).

In some embodiments, the hand-held device may comprise near-infrared spectroscopy (NIRS) sensors as part of a multimodal data acquisition.

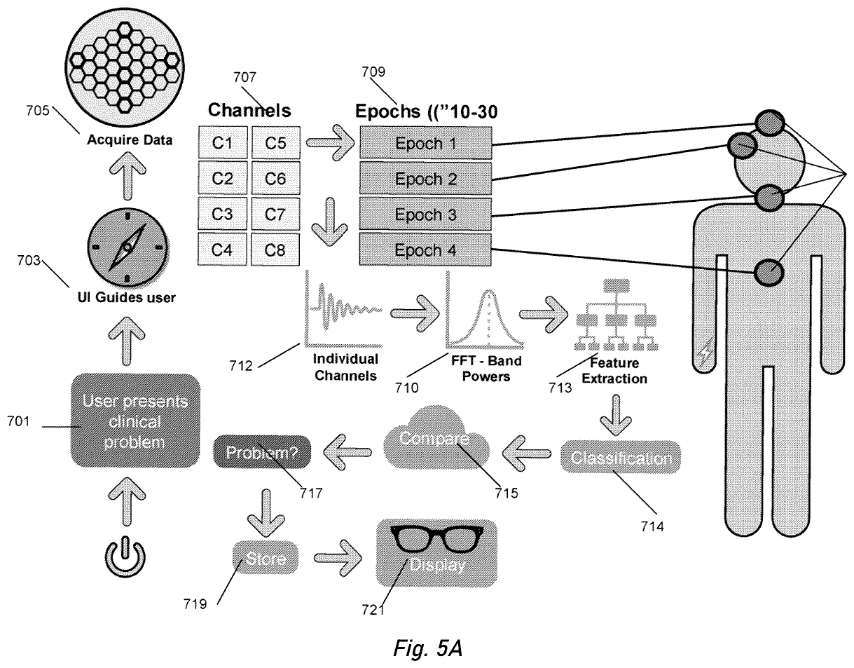

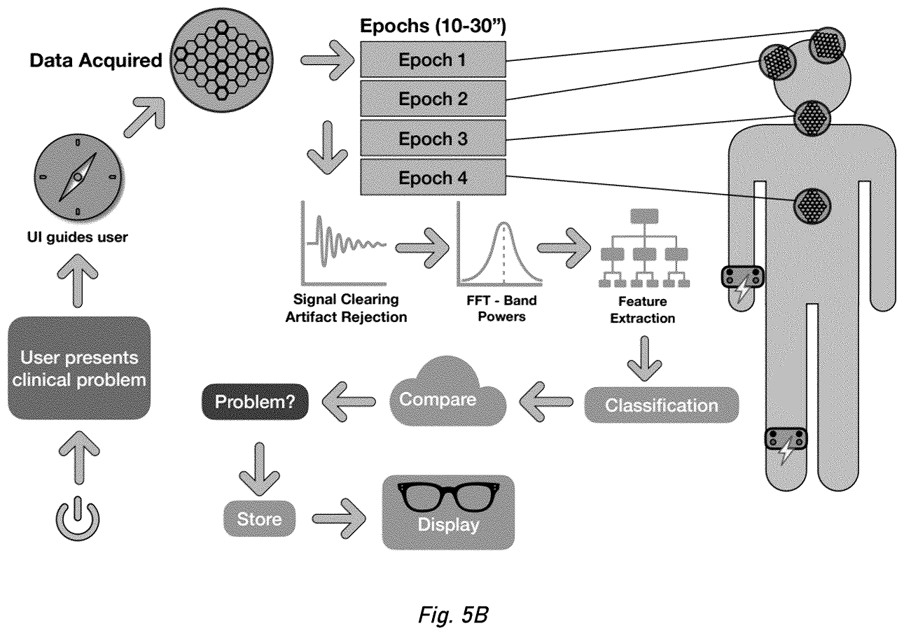

An aspect of some embodiments relates to interacting with a user of a hand-held device, for example to instruct the user regarding device positioning. In some embodiments, the hand-held device communicates with one or more remote devices, transferring the recorded data to the remote device and/or receiving input data from the remote device. In some embodiments, the remote device comprises a user interface (UI), optionally including a screen for displaying information to the user and/or for receiving input from the user. (FIGS. 5A-5B, 6A-6B, 7.) A user interface may be configured for example on a personal computer, a cellular phone, a tablet computer, a smart watch and/or other optionally portable device. In some embodiments, the user interface is configured as augmented reality glasses. (FIG. 5A-5B.) Optionally, the user wears the glasses while placing the hand-held device on the subject's body.

In some embodiments, the user interface is programmed to provide the user guidance and/or feedback regarding a position of the electrodes, a duration of recording, an anatomical region from which to record, or any combination thereof. In some embodiments, the user interface comprises imaging means (such as a camera) and circuitry configured to assess current electrode positions based on images acquired by the camera. Additionally or alternatively, the hand-held device comprises position indicators (e.g., optical marker(s), position sensor(s), proximity sensor(s), accelerometer(s), gravity sensor(s), rotation vector sensor(s), geomagnetic sensor(s), etc.) detectable by the system.

In some embodiments, the hand-held device is constructed to provide haptic feedback to the user, for example to direct electrode positioning, to indicate that recording was initiated, to indicate poor contact with the skin, to indicate potential existence of a pathology or irregularity, and/or other indications. In an example, the hand-held device comprises a vibrating mechanism, such as a spring based vibrating mechanism, which sets vibrations to thereby provide sensory feedback to the user holding the device.

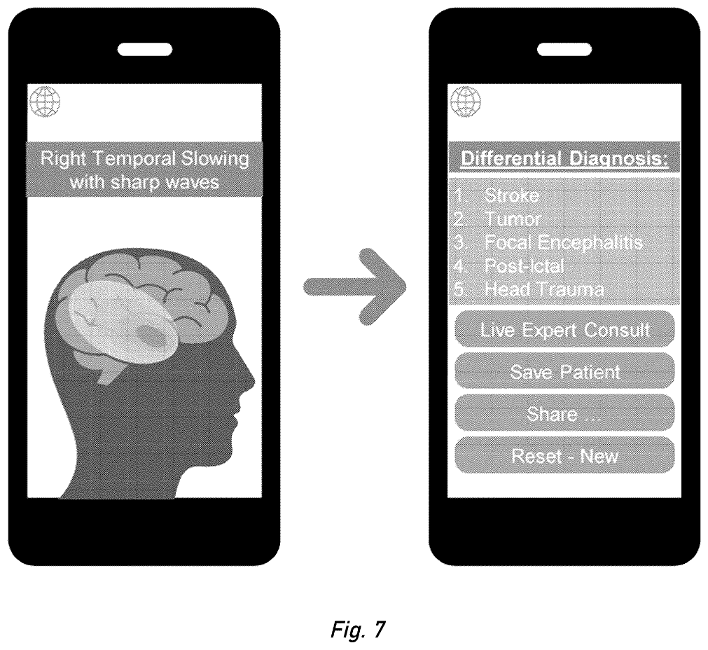

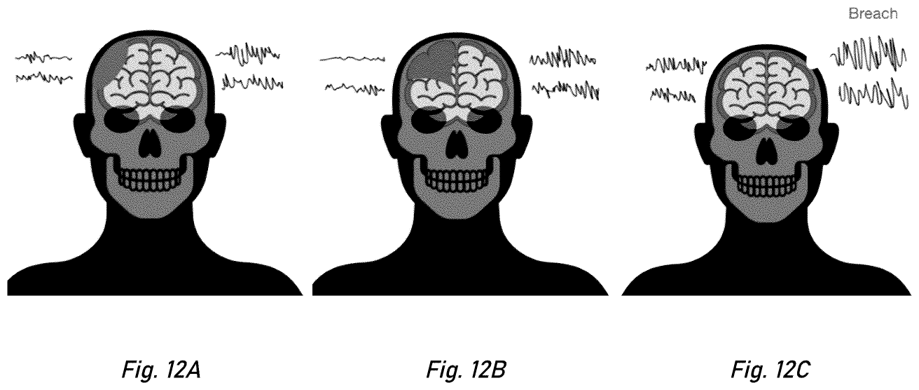

In some embodiments, the user interface is configured for processing of signals recorded from a subject, optionally in real time. In some embodiments, processing of the signals is carried out by detecting patterns in the recorded data (for example using feature extraction techniques), and comparing the patterns to pre-established patterns, such as patterns known from literature, patterns previously recorded from the same subject, patterns associated with a population, patterns associated with a certain physiological and/or pathophysiological state, optionally compared to a similar state in a large cohort (which may be considered as a normal baseline). Based on the detected patterns, an indication related to the subject condition is generated. (FIGS. 5A-5B.) Optionally, a differential diagnosis is generated and displayed to the user. (FIG. 7.) Examples of conditions which may be detected and/or assessed by devices and systems for example as described may include: encephalopathy (e.g. focal or generalized), status epilepticus, structural head injury, headaches (optionally for ruling out cerebral dysfunction), seizures, focal weakness or numbness, altered mental status, back pain, spinal cord pathology, multiple sclerosis, visual disturbance, hearing loss, toxic and/or metabolic encephalopathies and/or other conditions. (FIGS. 12A-12C, 14A-14C.)

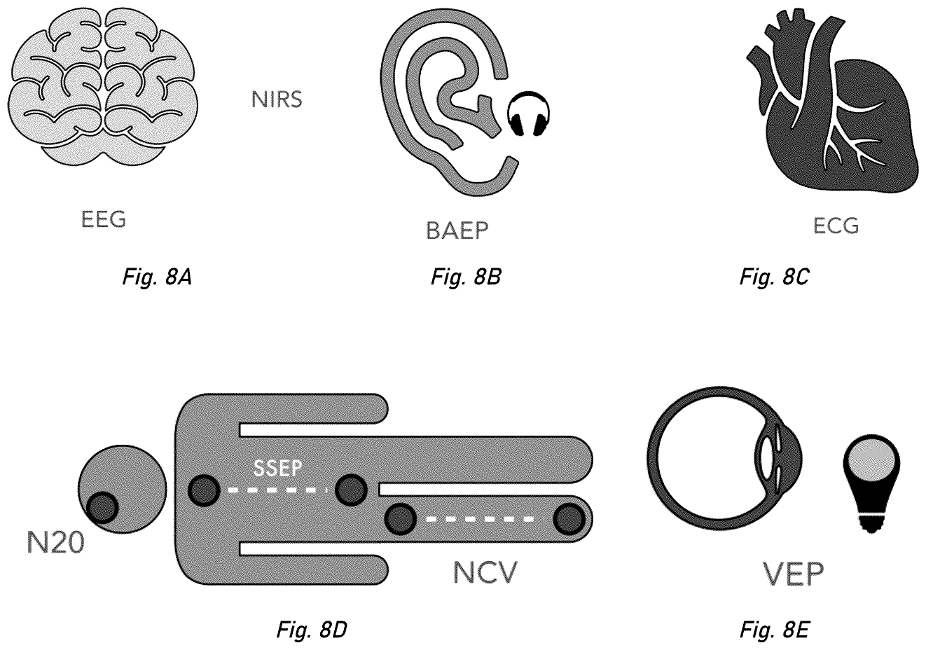

An aspect of some embodiments relates to detecting and optionally recording an evoked potential in response to stimulation using at least one hand-held device comprising a plurality of electrodes. In some embodiments, a stimulation is applied to a subject (e.g. an electrical stimulation; a visual stimulation; an audible stimulation) and a neural response to the stimulation is detected by the hand-held device (i.e. evoked potentials). In an example, electrical stimulation is applied to a limb, and the hand-held device is placed on the subject's scalp to record a somatosensory evoked potential (SSEP). (E.g., FIGS. 14A-14C.) In another example, visual stimulation is applied using a light source (Visual Evoked Potential-VEP). In another example, audible stimulation is applied via headphones or the like (for Brainstem Auditory Evoked Potentials-BAEP). (FIGS. 8A-8E.) Stimulating and measuring may be carried out via a pair of hand-held devices (e.g. using one device to apply the stimulation, and the other to measure the response) or alternatively using any stimulating device (e.g., sleeve, clip comprising suitable circuitry) and a hand-held device to measure the response. In some embodiments, stimulation is applied to a distal limb portion, and recording is performed from a plurality of positions along the ascending neuro-axis. For example, the hand-held device is positioned at a plurality of locations along the neural path (e.g., the peripheral nerve, portions of the plexus, spinal cord, scalp). Optionally, a location of transmission interruption is detected, indicating a possible pathology. In any embodiment, the subject may be a human patient.

In some embodiments, detection of evoked potentials may provide for identifying a pathological source or condition.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

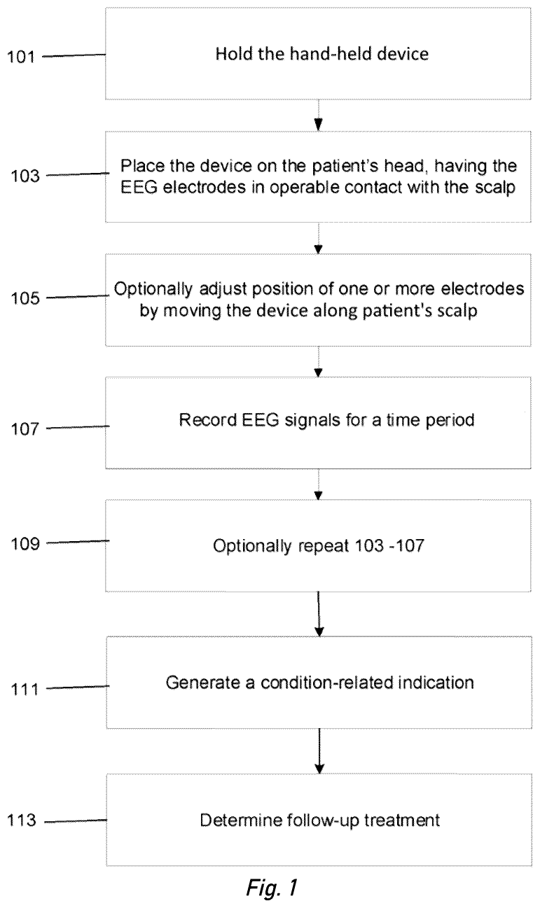

Referring now to the drawings, FIG. 1 is a flowchart of a general method for recording EEG signals using a hand-held device, according to some embodiments.

In some embodiments, a method for example as described herein is implemented for diagnosing (or otherwise contributing to diagnosis) of a subject during emergency situations, when a fast, dynamic assessment of the subject condition is advantageous. A method for example as described herein may be carried out in a non-hospital setting (such as for emergency medicine providers, military medicine, ambulance, rural clinics and the like), or, alternatively, in a clinical setting, such as in an emergency room, physician clinic and/or generally at any situation in which a rapid and simple assessment of the subject condition is advantageous, for example in assisting diagnostic and/or therapeutic decision making. Additionally or alternatively, a method for example as described herein is implemented during non-emergency situations, for example, as a protocol for standard assessment and screening of a subject for predetermined conditions, such as at a family practice clinic.

A method as described herein may be especially advantageous for identifying conditions related, for example, but not limited to: diffuse or focal encephalopathy (of any etiology-such as, but not limited to: ischemic stroke, brain hemorrhage, brain tumor); status epilepticus, seizures, interictal epileptiform discharges (IEDs); assisting in ruling out electrophysiological changes in cases of structural brain injury, headaches; assessing for electrophysiological disturbance in cases of focal weakness or numbness, altered mental status, back pain, spinal cord pathology (tumors, abscess, myelopathy of other causes such as multiple sclerosis), visual disturbance (either of central origin via occipital EEG, or pre-cortical-via VEP), focal hearing loss, toxic or metabolic encephalopathy, assessment after anoxic brain injury (such as in, but not limited to: drowning, prolonged resuscitation), neurodegenerative disease (periodic patterns, P300 latency) and/or other conditions.

At 101, a user (e.g., a physician, registered nurse, nurse practitioner, physician assistant, paramedic, emergency medical technician (EMT), military medic, other care giver or in some embodiments, the subject themselves) holds the hand-held device in their hand. For example, the device is in the form of a matrix comprising flexible substrate, such as a spongy material, formed into a substantially spheroid 3-dimensional shape, and the user grips the spheroid device. The device may be configured to receive any electrophysiological signal including, but not limited to EEG. Optionally, the device comprises a plurality of EEG electrodes positioned such that upon placing of the device on the subject's head, the surface of the devise conforms to the conformation of the subject's head, and the electrodes come into operable contact with the scalp. As an additional advantage, flexible material, such as, e.g., a spongy material, makes contact between the hand-held device and the subject gentle, minimizing disturbance of the subject's head, reducing risk of aggravating the subject's condition. For instance, in the case of a suspected neck injury, the flexibility of the device material ensures contact forces between the device and the subject is as mild as possible and will not disturb the subject's head position.

At 103, the user places the device on the subject's head, ensuring that at least some of the electrodes operably contact the scalp. In some embodiments, contact is made through subject's hair, for example by the device including electrodes having pins, spikes or other suitable design for passing through hair to operably contact the scalp.

At 105, the user optionally adjusts a position of one or more of the electrodes relative to the scalp, for example by moving the device relative to the subject's head. In some embodiments, setting and/or adjusting a device position is according to guidance, for example automatic guidance provided to the user by a user interface of the hand-held device, such as via a dedicated cellular phone or tablet application. In some embodiments, guidance provided to the user includes instructions for spatially positioning the electrodes, a minimal duration for recording EEG at each position, and/or requests for input (such as personal subject data and/or data relating to a current condition or symptom and/or relevant past medical and/or surgical history) from the user.

In some embodiments, the user interface is programmed to provide guidance in accordance with input data inserted by the user, for example, the user inserts an estimation of a medical condition, a list of symptoms exhibited by the subject, and/or other input data, and guidance (e.g., regarding device positioning) is selected according to the input data. Additionally or alternatively, a scanning mode is implemented, for example in which no pre-assumed condition is inputted, and the user places the device at multiple different spatial scalp positions while scanning for potential electrophysiological changes (such as EEG patterns) which may be indicative of the subject's condition.

In some cases, an experienced and/or expert user (e.g., a neurologist) may place the device directly at a selected position, which the user suspects as informative to their diagnostic purposes, for example at a position which detects a source of aberrant neural activity or conduction.

At 107, EEG signals are recorded for a time period of, for example, between 5-30 seconds, such as 10 seconds, 20 seconds, 25 seconds or intermediate, longer or shorter time periods. The duration may, without limitation, be less than 1 second, about 1 second, about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, about 31 seconds, about 32 seconds, about 33 seconds, about 34 seconds, about 35 seconds, about 36 seconds, about 37 seconds, about 38 seconds, about 39 seconds, about 40 seconds, or greater than 40 seconds. Optionally, the duration is influenced by the quality of the acquired data. In some embodiments, at each change of position, calibration of the device is performed, for example to set operation settings according to the current electrode positions. In some cases, device calibration may add between 2-6 seconds to each recording session. For example, device calibration may add less than 2 seconds, about 2 seconds, about 3 seconds, about 4 seconds, about 5 seconds, about 6 seconds, or longer to each recording session.

Optionally, the recorded data is transferred to a device memory or database. In some embodiments, the recorded data is communicated, optionally in real time, to a medical center, a physician's clinic, cloud memory, and the like.

At 109, optionally, one or more of steps 103 through 107 are repeated. In some embodiments, the user adjusts a position of the device with respect to the subject's head, so as to position at least some of the electrodes at different spatial locations relative to the previous spatial locations recorded from the subject. Optionally, the user skims through several (e.g., 2, 3, 4, 5, 10 or intermediate, larger or smaller number) different spatial positions.

At 111, in some embodiments, a subject condition-related indication is generated. In some embodiments, the condition-related indication is generated by processing the EEG or other electrophysiological data collected over the plurality of recording sessions.

In some embodiments, the condition-related indication is generated in accordance with other input data, including, for example: personal subject information (e.g., age, weight, height, past medical and/or surgical history); symptoms exhibited by the subject; events associated with a current subject condition (e.g., the subject is a person who was involved in vehicular collision, the subject fainted); an assumed medical diagnosis; vital signs such as body temperature, heart rate, blood pressures, respiration rate, pregnancy and/or other data. Optionally, the vital signs assist in determining a subject's condition, for example, slow heart rate and high blood pressure may support existence of increased intracranial pressure; low blood pressure with a high fever may indicate sepsis; etc.

In some embodiments, processing of the collected EEG data is carried out using pattern recognition techniques. Optionally, patterns identified in the collected data (e.g., using feature extraction techniques) are compared to pre-established patterns (e.g., patterns pertaining to the same subject, normal physiological and pathophysiological patterns as collected in large cohort population, literature-based patterns, and/or other) for generating the condition-related indication.

At 113, in some embodiments, the user determines how to continue treating the subject. Optionally, the decision is made in accordance with the generated differential diagnosis. Optionally, the user interface automatically suggests a plan for continuing investigation and/or treatment. In some cases, the user continues treating the subject; additionally, or alternatively, the subject is transferred to a medical center and/or to other medical personnel to receive additional treatment. In an example, the follow-up treatment plan includes imaging the subject, for example via CT/MRI, while indications obtained by the hand-held device may assist is determining a specific anatomical region to be imaged (thereby potentially saving time, money, and/or discomfort to the subject).

In a non-limiting prophetic example, a user, e.g., a paramedic, arrives at the scene of an automobile collision. A subject injured in the collision is undergoing emergency care at the scene. The user optionally inputs on a mobile UI that the circumstance is physical trauma. The user places the hand-held device on the subject's scalp such that electrodes are in operable contact with a first location on the subject's scalp. The device reads EEG from the electrodes at the first location for 20 seconds. The user than moves the device to a second location on the subject's scalp. The device reads EEG at the second location for 20 seconds. The user than moves the device to a third location on the subject's scalp. The device reads EEG at the third location for 20 seconds. The user than moves the device to a fourth location on the subject's scalp. The device reads EEG at the fourth location for 20 seconds. The plurality of electrodes, and series of four positions along the subject's scalp indicate a significant serial high-amplitude arch-like waveform when at location three. From the circumstances and EEG data, the system generates an indication that the subject has a suspected skull breach at location three, and displays the indication on a UI display.

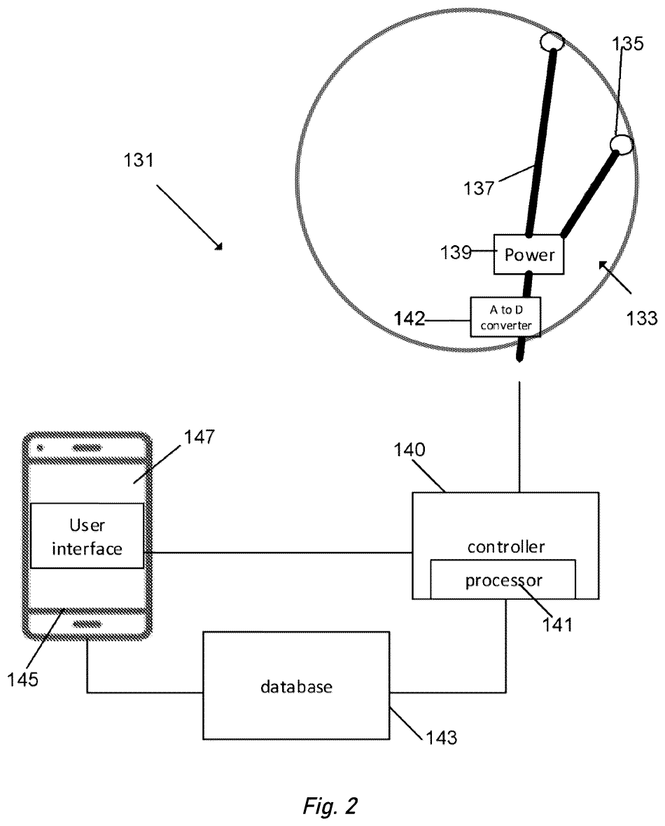

FIG. 2 is a diagram of a basic system for recording EEG signals using a hand-held device, according to some embodiments.

In some embodiments, system 131 comprises a hand-held device 133, including a plurality of electrodes 135 disposed on an outer surface of the device, for example 2, 3, 4, 5, 6, 7, 8, 9, 10, 15, 20 electrodes or intermediate, larger or smaller amount disposed on an outer surface of the device. Optionally, the electrodes are EEG electrodes. In some embodiments, device 133 is designed to fit in a user's hand, for example, being formed as a ball, sphere, hemisphere, or bowl/cup shape. In another example, electrodes may be placed by a user onto the flexible surface of the device, the electrodes having an adhesive surface, and are applied to the surface of the device as stickers that the user can place, remove, and replace as required. In some embodiments, the electrodes may be single-use and/or disposable.

Optionally, the user adjusts electrode position by moving the device. Optionally, the user adjusts electrode contact with a surface of a subject's body, such as the subject's scalp, for example by pressing down on and/or slightly lifting up one or more selected electrodes relative to the subject's scalp and/or adjusting the contact angle.

In some embodiments, electrodes 133 are connected via circuitry 137 suitable for electrically activating the electrodes, such as by connecting the electrodes to a power source 139 (e.g., a battery, for example a 5-volt battery). In some embodiments, circuitry 137 is configured as thin wiring, optionally embedded within the substrate material.

In some embodiments, the power source is integrated in the hand-held device. Alternatively, in some embodiments, the power source is positioned separate from the device, optionally powering it remotely (via wired and/or wireless communication).

In some embodiments, device 133 comprises circuitry for digitizing the recorded signals, for example an analog to digital converter 142.

It is noted that passive and/or active electrodes may be used, affecting the powering requirements.

In some embodiments, device 133 (optionally via circuitry 137) is connected to or comprises a controller 140. Additionally or alternatively, control of the hand-held device is via a computational unit of a personal electronic device such as a designated device, a cell phone or a tablet. Functions as described herein with respect to the controller may be carried out by the computational unit, in replacement and/or addition to the device controller.

In some embodiments, controller 140 controls activation of electrodes 133. In some embodiments, controller 140 comprises a processor 141 programmed to analyze the recorded data (e.g., EEG or other). Optionally, the processor is programmed to apply various algorithms on the collected data, for example, to carry out deep neural network analysis of the data.

In some embodiments, controller 140 is in communication with a database 143. In some embodiments, recorded data is communicated to the database and stored there. In some embodiments, database stores pre-established patterns which the recorded data is compared to. In some embodiments, database 143 stores protocols for device activation, for example including timing of recording sessions and/or electrode positioning information. In some embodiments, database stores recorded data of a plurality of subjects. Optionally, the recorded data is linked to a subject condition, medical indication and/or differential diagnosis.

In some embodiments, system 131 comprises a user interface 145. In some embodiments, user interface 145 is configured on a personal device, optionally a hand-held device, such as cellular phone or tablet, a personal watch (e.g., smart watch), and/or other wearable or other in-network environmental computing system.

In some embodiments, user interface 145 comprises a display 147. Optionally, the interface (for example via a screen display, such as a touchscreen display) is configured for receiving input from the user (e.g. subject personal information, symptoms, vital signs, related event and/or other). Optionally, display presents visual feedback to the user, for example guidance for positioning electrodes, a graphical analysis of recorded signals, proposed diagnosis, proposed plan for continuing treatment, and/or other data. In some embodiments, user interface 145 provides audible feedback (e.g. voice instructions to the user). In some embodiments, user interface 145 provides tactile feedback to the user, for example, the device (e.g. cell phone) vibrates when an electrode is properly placed.

It is noted that in some embodiments, circuitry (including, for example, signal amplifier, converter, communication components and/or other components) is separable or provided separately from the device itself. Optionally, circuitry is configured at the user interface and/or other remote device.

In some embodiments, the user interface is configured to receive input from the user in the form of manual commands (e.g., by pressing a button); voice commands; text commands and/or other.

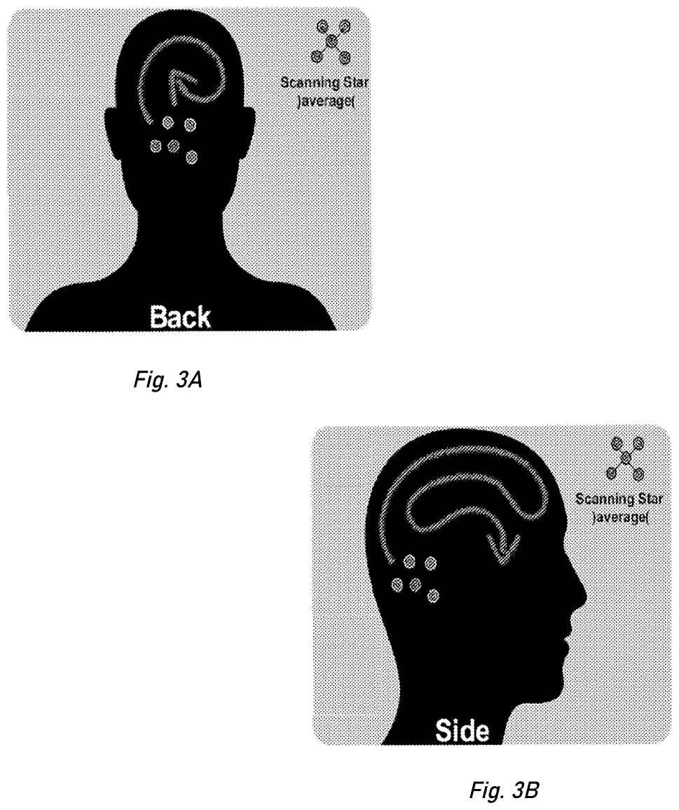

FIGS. 3A-3B schematically illustrate examples of positions of a hand-held device comprising electrodes on a subject's head, according to some embodiments. The device may comprise five electrodes in a “scanning star” shape and/or a user may position the device in five or more locations forming a “scanning star” shape, as shown in FIGS. 3A-3B.

In some embodiments, electrodes are positioned spread apart from each other. Alternatively, electrodes are clustered together.

FIG. 3A illustrates a position in which the device electrodes are arranged to measure at a posterior position, according to some embodiments. Optionally, the electrodes are arranged in a cross or star pattern for average reference montage. A user may position and reposition the hand-held device along the subject's scalp according to a predetermined pattern, illustrated, for non-limiting example, by the curved arrow shown in FIG. 3A. It is noted that the positions are provided only as examples and that other positions and/or electrode layout configurations are contemplated as well.

FIG. 3B illustrates a position in which the device electrodes are arranged to measure as a weighted average reference montage from either side of the head, according to some embodiments. Optionally, the electrodes are arranged in a cross or star pattern. A user may position and reposition the hand-held device along the subject's scalp according to a predetermined pattern, illustrated, for non-limiting example, by the curved arrow shown in FIG. 3B. It is noted that the positions are provided only as examples and that other positions and/or electrode layout configurations are contemplated as well.

Advantageously, having a plurality of electrodes set at a fixed position relative to each other electrode allows for Laplacian spatial filtering methods of signal processing.

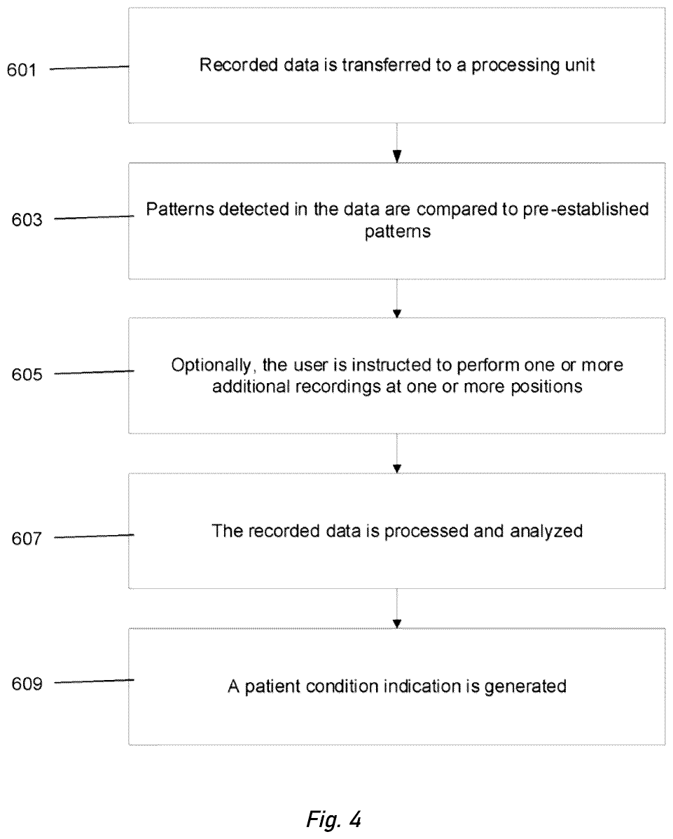

FIG. 4 is a flowchart of a method of generating a subject condition indication based on data acquired by a hand-held device, according to some embodiments.

At 601, in some embodiments, data recorded by the electrodes (such as EEG signals) is transferred to a processing unit, for example including a device processor, a remote server, a cloud server, and/or other. At 603, patterns detected in the recorded data are compared to pre-established patterns, for example, patterns known in literature, patterns previously collected from the same subject; patterns collected from a plurality of subjects, and optionally associated with the same condition, diagnosis and/or symptoms. In some embodiments, processing of the data includes identifying a local source of the bioelectrical signal based on the spatial position of the electrodes at the time of recording.

In some embodiments, a remote device or server comprises a computer in a subject's room, an ambulance, and/or other setting. In some embodiments, the device and/or user interface are configured to communicate with a database such as a hospital patient database (i.e. the electronic medical record—EMR).

At 605, optionally, the user is instructed to perform one or more additional recordings at one or more scalp positions. Optionally, the one or more positions differ from a previous recording position by a spatial location of at least one of the electrodes. In some embodiments, guidance is provided to the user, optionally via the user interface, to assist in positioning and/or instruct regarding a duration of recording, properties of the signal being currently recorded, improvement of contact with the scalp and/or others.

In some embodiments, a protocol (or sequence) according to which recording is carried out is set based on user input, including for example current symptoms of the subject, an associated event (e.g., a fall or a vehicular collision, etc.), personal patient data, vital signs, etc. In some embodiments, a protocol according to which recording is carried out is set based on an initial recording, optionally one in which the user scans the subject's head to detect and localize the approximate source of the abnormal signal. Optionally, real-time analysis of the first recording allows for categorizing the subject condition into one of several categories, according to which diagnosis options are suggested and/or a protocol for additional measurements is proposed.

At 607, in some embodiments, the additional data that was recorded is processed and then analyzed, and at 609, a subject condition indication is generated.

In some embodiments, analysis of the data for reaching a condition related indication is performed in real time, for example within about 30 seconds, about 1 minute, about 5 minutes, about 10 minutes or intermediate, longer or shorter time periods from an initial recording.

FIGS. 5A-5B are graphical flow diagrams of exemplary methods for processing data acquired by a hand-held device, according to some embodiments.

In FIG. 5A, at 701, optionally, a user presents a clinical problem, optionally inputting the problem via the user interface. At 703, in some embodiments, the user is guided, optionally by the user interface, to acquire data 705 (such as, but not limited to, EEG signals). At 707, in some embodiments, a plurality of recording channels (e.g., 8 channels as described in FIG. 5A) are used for measuring during, for example, four different epochs 709, each epoch performed for a time period of between, for example, about 10-30 seconds. In some embodiments, recordings are performed not only from a head of the subject, but also from other anatomical locations, e.g., neck, heart, for example as shown at 711. Optionally, different data types are collected from various locations (e.g., electrocardiogram (ECG) from the heart, evoked potentials, nerve conduction velocity (NCV), etc.).

In some embodiments, an anatomical position is identified, and a recording protocol and/or analysis protocol are selected based on the identified position. In some embodiments, the position is identified via imaging, for example using an imager of the user interface device (e.g., cell phone camera). Additionally or alternatively, the position is identified based on indications from one or more position sensors of the device. Optionally, interpreting of the recorded data is based on the electrode positions, for example as indicated by the position sensors.

In some embodiments, based on the anatomical location from which recording is performed, automatic calibration of the device occurs. In an example, the measurement units are matched to the type of signal being recorded (e.g., for measuring ECG from the heart, the device switches to measurement in millivolts, for measuring EEG from the brain, the device switches to microvolts).

Various methods may be implemented for reducing noise during and/or following recording. In some embodiments, noise is reduced during recording, by instructing the user to apply a firm pressure while pressing the electrodes against the scalp, which may decrease impedance. In some embodiments, noise is reduced with the aid of active electrode sensors which apply minor current in a certain spot, and recording the change in the minor currents, for example in near-field and far-field electrodes. Signal noise may be reduced using a Laplacian strategy using an array of sensor electrodes at a fixed location relative to each other sensor electrode.

In some embodiments, noise is reduced (or removed) from the recorded data, for example by filtering the data, e.g., using a 1-50 Hz bandpass filter, or other high and/or low pass filter configurations. In some embodiments, noise is reduced by average summation of the recorded signals, thus reducing non-persistent signals (such as random or temporary frequency patterns) and amplifying common, repetitive patterns. In some embodiments, a notch filter is applied (compatible with the local power grid frequency—50 or 60 Hz respectively).

In some embodiments, noise reduction involves isolating the channels recorded by the device from the user's bioelectricity effects and/or other ambient electromagnetic forces.

In some embodiments, the recorded data is analyzed. In some embodiments, analysis is carried out by filtering the data recorded at the plurality of individual channels 712; applying feature extraction 713; and classification 714 to identify patterns. Optionally, algorithms such as FFT 710 are applied to convert the recorded signals to the frequency domain. Then, in some embodiments, predefined EEG bands such as alpha, theta, beta, delta are sorted, and their power is measured. Furthermore, presence of other specific morphologies and/or artifacts such as triphasic waves, epileptiform sharp waves, spikes, and/or other waveforms are detected. In some embodiments, waveforms detected are evaluated for rhythmicity and/or periodicity as their significance differs from a sporadic distribution of the same waveforms.

In some embodiments, at 715, the detected patterns are compared to pre-established patterns. At 717, based on the processed data and/or additional input, a clinical problem (indication) is reached. Optionally, the acquired data confirms the initial problem suspected by the user or may offer insight regarding the appropriate differential diagnosis in view of presenting signs, symptoms and/or acquired electrodiagnostic/electrophysiological data.

At 719, the collected data and/or associated clinical indication is optionally stored (e.g., in a device memory, clinical database and/or other). At 721, the clinical indication is presented to the user, for example on a user interface screen.

Some examples of significant wave patterns and a condition associated with these patterns may include: increased beta activity may be indicative of alcohol ingestion, use of benzodiazepines or barbiturates; ictal or interictal epileptiform activity indicates tendency for focal or generalized seizures; triphasic waves may be indicative of metabolic derangements (e.g., liver or kidney failure); increased delta-theta power with subsequent decreased alpha-beta activity in a focal region may be indicative of a stroke (either ischemic or hemorrhagic), but not limited.

As shown in FIG. 5B, the subject may optionally be fitted with an electrical stimulation device, for example in the form of a wrist bracelet, ankle cuff, or both a wrist bracelet and an ankle cuff. The hand-held device may be positioned at different locations on the subject's body, recording somatosensory evoked potentials for an epoch at each location, thereby resolving, e.g., a location of a spinal cord injury.

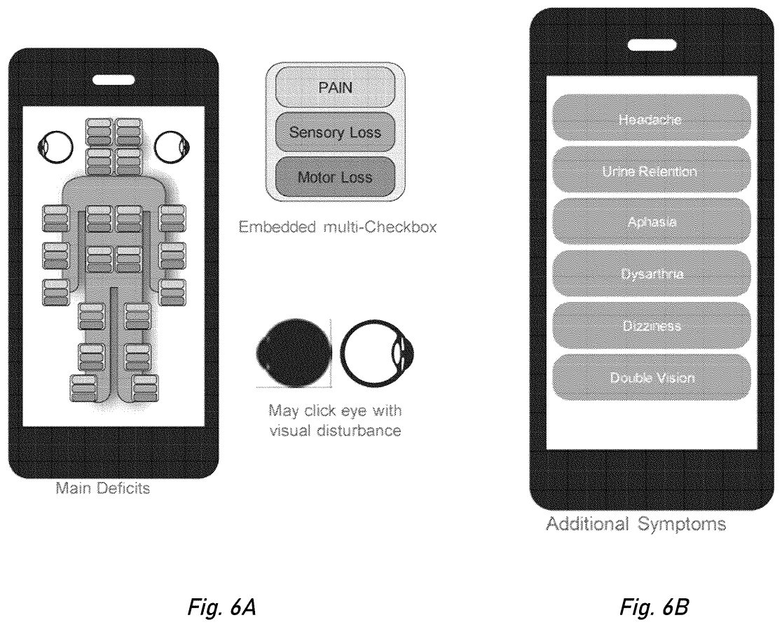

FIGS. 6A-6B are examples of user interface screens of a hand-held device used to control a hand-held electrode array, according to some embodiments.

The following are examples of print screens of a user interface, for example configured as a cellular phone application. In some embodiments, the user interface is designed and configured to receive input from the user and/or subject; to guide the user during data acquisition; and/or to provide feedback, such as a differential diagnosis, options for continuing treatment, and/or other output.

In the example of FIG. 6A, a chief complaint screen is shown, including check boxes for indicating one or more of: pain, sensory loss, motor loss, vision loss and/or other.

In the example of FIG. 6B, a screen for inputting additional symptoms is presented. Optionally, the options listed are tailored to the selections in the chief complaint screen. For example, if motor loss or weakness is indicated for both legs, the proposed list of symptoms is set to include “urine retention” and/or “back pain”. In another example, in the case of right arm and leg weakness, the system will inquire regarding symptoms such as “aphasia”, “headache”.

In the examples of FIG. 7, one or more findings are presented. Optionally, a differential diagnosis and/or other condition-related indication are presented. In some embodiments, the interface allows a user to consult an expert, save the data, share the data (e.g., with a physician), and/or reset/start over. In an example, data is sent from an emergency medical service to a neurologist, allowing a stroke team at the hospital to optimize care for the subject. For example, in case there is suspicion for a large vessel occlusion, the emergency medical team may be routed with the subject to a suitable hospital with brain catheterization competence, therefore potentially saving valuable time and improving subject outcome, thus reducing hospital and post-hospital costs.

FIGS. 8A-8E show various electrophysiological information types which may be measured by the systems and device of the present disclosure. Such electrophysiological information includes, but not limited to: electroencephalography (EEG) (FIG. 8A), electrocardiogram (ECG) (FIG. 8C) data; nerve conduction velocity (NCV) data (FIG. 8D); visual evoked potentials (VEP) (FIG. 8E); brainstem auditory evoked potentials (BAEP) (FIG. 8B); somatosensory evoked potentials (SSEP); or any combination thereof. The device and systems of the present disclosure may also acquire near infra-red spectroscopy (NIRS) data and/or other diagnostic data. NIRS requires use of additional IR source and sensor, while all evoked potential modalities (VEP, BAEP, SSEP) require stimulation, which may come from a stimulation module (strobe light, sound, electrical stimulation).

FIG. 9 schematically illustrates that electrodes of the hand-held device may form an electrode array, with the electrodes “tiled” at a substantially fixed distance from each other (upper left) for resolving spatial EEG information, that the device may further incorporate electrocardiogram (ECG) and/or other electrophysiological signal data (bottom left), that EEG samples may be drawn from multiple locations on subject's scale (center), and that scalp EEG signal data may be combined with SSEP signal (right).

FIGS. 10A-10B schematically illustrate exemplary spatial arrangements of electrodes of the hand-held device. FIG. 10A shows an example without NIRS, and FIG. 10B shows an example with NIRS. The electrodes may be Laplacian. Setting the electrodes at a fixed distance relative to each other electrode allows for normalizing resistance and potential values across the distance. This fixed distance allows for resolving and comparing differences in signal amplitude between two different locations on a subject's body.

FIG. 11 is an example of the hand-held device of the present disclosure. In this example, the subject is a human patient. A user, which may be a medical professional or the subject, grasps the device, which has an array of electrodes disposed on the surface, and contacts it with the subject's scalp. The flexible substrate allows that the electrode array conform itself to the contour of the subject's scalp, thereby operably contacting many or all of the electrodes to the subject's scalp. The array of electrodes may send EEG data via different channels to a computer, which processes the data.

FIGS. 12A-12C are illustrations of expected EEG signal measured at different locations on a subject's scalp for different medical conditions, namely subdural hematoma (FIG. 12A), large vessel occlusion (LVO) (FIG. 12B), and skull breach (FIG. 12C). Each condition produces a distinct EEG signal modality and spatial presentation of such modality relative to the site of injury/condition. For instance, LVO tends to cause severely attenuated EEG signal as read at locations on the subject's scalp at any surface proximate to blood vessels distal from the site of occlusion. A severe occlusion will result in large affected surface area and/or more severely depressed EEG signal. Moving the hand-held device of the present disclosure around the subject's scalp allows a user to ascertain the qualities of the signal, and location and area affected.

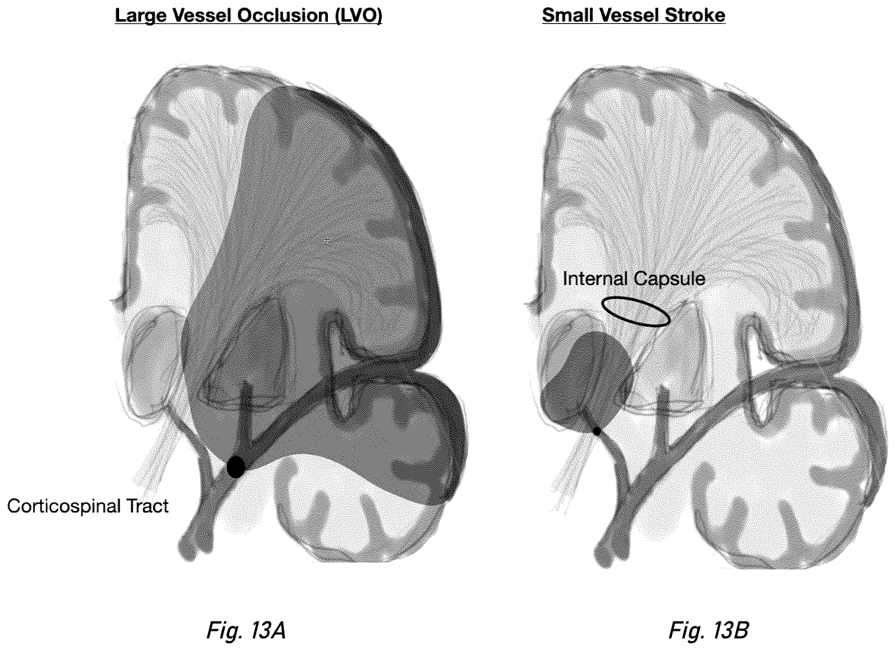

FIGS. 13A-13B are exemplary illustrations of section of brain tissue. A small vessel stroke (FIG. 13B) affecting a more distal blood vessel will tend to affect a smaller area of brain tissue. An LVO (FIG. 13A), affecting a larger, more proximal blood vessel will tend to affect a large area of downstream brain tissue. The hand-held device of the present disclosure, having an array of electrodes at a fixed distance relative to one another, and movable to different locations across a subject's scalp, allows for quickly ascertaining the severity and area of stroke-indicative EEG signal.

FIGS. 14A-14C are exemplary illustrations of use of the hand-held device of the present disclosure on a human-patient subject with different body injuries. The subject, in this example, is fitted with an evoked stimulus device on the subject's left ankle. In FIG. 14A, the subject has a brain injury. A user takes hand-held device successive readings at left leg, lower spine, cervical spine, and on the subject's scalp. Normal electrophysiological signal is readable at each location except the subject's scalp, indicating the subject has a brain injury but not a spinal cord injury. In FIG. 14B, the subject has a cervical spine injury. Accordingly, evoked signal from the subject's ankle is readable at the leg and lower spine, but is blocked at the cervical vertebra. In FIG. 14C, the subject has a lower spine injury. Accordingly, in this example, the evoked signal from the subject's ankle is readable at the leg, but is blocked at the lower spine and is not readable at any more rostral location from the injury site, along the subject's spine and up to the brain. Thus, the location of injury may be quickly resolved.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Embodiments of the present disclosure may be further understood by reference to the following clauses:

Clause 1. A hand-held device for electrophysiology data recording from a subject, comprising:
a flexible substrate having a plurality of electrodes mounted onto an external surface of the substrate; and
circuitry configured to acquire and process electrophysiology data signals recorded from the subject by the electrodes and for communicating the recorded electrophysiology data signals to a remote device.

Clause 2. The device according to clause 1, wherein the flexible substrate comprises an electrically insulating material.

Clause 3. The device according to clause 2, wherein the flexible substrate comprises a reverse surface lined with an electrically insulating lining.

Clause 4. The device according to any of clauses 1-3, wherein the device is formed as a 3-dimensional shape selected from the group consisting of: sphere, hemisphere, spheroid, polyhedron, cylinder, torus, toroid, and hyperboloid.

Clause 5. The device according to any of clauses 1-4, wherein the circuitry includes an analog to digital converter, an amplifier, and a communication module.

Clause 6. The device according to any of clauses 1-5, each of the two or more electrodes are at a fixed distance relative to each other electrode on the external surface of the substrate.

Clause 7. The device according to any of clauses 1-6, wherein the electrodes are dry electrodes, each electrode comprising a plurality of pins long enough for contacting the subject's scalp through hair.

Clause 8. The device according to clause 7, wherein the pins protrude at least 2 mm away from the external surface of the substrate.

Clause 9. The device according to any of clauses 1-8, wherein the circuitry comprises a controller programmed to control recording by the electrodes based on input received from a remote device.

Clause 10. The device according to any of clauses 1-9, wherein each electrode comprises at least one spring positioned between the electrode and the external surface of the substrate, which provides resistance in response to pressure applied onto the electrode.

Clause 11. The device according to any of clauses 1-10, comprising one or more optical markers positioned on the external surface, the optical markers detectable by the remote device.

Clause 12. The device according to any of clauses 1-11, further comprising one or more near-infrared spectroscopy sensors on the external surface of the substrate.

Clause 13. The device according to any of clauses 1-12, wherein the electrophysiological data is selected from any of: EEG, ECG, NCV, BAEP, and SSEP.

Clause 14. The device according to any of clauses 1-12, wherein the electrophysiological data is EEG.

Clause 15. The device according to any of clauses 1-13, wherein the electrodes are arranged in an x×y matrix, wherein x and y are, independently, integers from 1 to 10.

Clause 16. The device according to clause 15, wherein the matrix is a 5×5 matrix.

Clause 17. A system comprising:
a device according to any of clauses 1-16, and
a remote device including a user interface, the user interface configured for receiving input data and for generating a subject condition related indication based on the input data and on the electrophysiological signals recorded by the electrodes.

Clause 18. The device according to clause 17, wherein the circuitry is programmed to communicate with the remote device and wherein the remote device is programmed to control recording by the electrodes.

Clause 19. The system according to either of clauses 17 or 18, wherein the input data includes a current position of the electrodes with respect to the surface of the subject's body.

Clause 20. The system according to either of clauses 17 or 18, wherein the input data includes a current position of the electrodes with respect to the subject's head.

Clause 21. The system according to any of clauses 17-20, wherein the input data includes one or more of: vital signs, symptoms exhibited by the subject; an assumed medical condition, an associated event which the subject was involved in, and the subject's medical and surgical history.

Clause 22. The system according to any of clauses 17-21, wherein the user interface comprises a screen for presenting operating instructions to a user relating to the position of the electrodes with respect to the surface of the subject's body.

Clause 23. The system according to any of clauses 17-22, wherein the user interface comprises a screen for presenting operating instructions to a user relating to the position of the electrodes with respect to the subject's head.

Clause 24. The system according to any of clauses 17-23, wherein the remote device comprises a smart phone or a tablet computer.

Clause 25. The system according to any of clauses 17-24, wherein the remote device comprises:

an imager; and circuitry configured to identify a position of the electrodes with respect to the subject's anatomy from images acquired by the imager.

Clause 26. The system according to any of clauses 17-25, wherein one or both of the hand-held device and the remote device are configured to transfer and/or receive data to and/or from a medical center database.

Clause 27. The system according to clause 26, wherein the data includes the subject condition related indication generated by the system.

Clause 28. The system according to either clause 26 or 27, wherein the data includes follow-up instructions generated based on the subject related condition indication.

Clause 29. The system according to clause 28, wherein the follow-up instructions include recommendations for imaging the subject and/or for the type of imaging modality required and/or the anatomical region deemed relevant for imaging.

Clause 30. The system according to any of clauses 17-29, wherein the remote device is configured for generating the subject related condition indication within 3-6 minutes of recording the electrophysiological signals

Clause 31. The system according to any of clauses 17-30, wherein the user interface is configured as augmented reality glasses to be worn by the user.

Clause 32. The system according to clause 31, wherein the glasses are configured to present to the user one or more of: instructions for positioning the hand-held device on the subject; a duration for measuring; currently recorded data; subject condition related indications.

Clause 33. A kit comprising:

a set of two hand-held devices according to any of clauses 1-16, wherein at least one of the devices further comprises a stimulation module for applying an electrical, visual and/or audible stimulation to the subject.

Clause 34. A method for recording an EEG of a subject using a hand-held device comprising a plurality of electrodes, comprising:
contacting the plurality of electrodes with the subject's scalp, resulting in electrically conductive contact between the subject's scalp and at least two electrodes; and
recording EEG signals.

Clause 35. The method according to clause 34, further comprising obtaining an automatically generated indication related to a condition of the subject, the indication at least partially based on the recorded EEG signals.

Clause 36. The method according to either clause 34 or 35, wherein the recording is for a time period of between 5-30 seconds for each recording position or epoch.

Clause 37. The method according to any of clause 34-36, comprising adjusting a position of the hand-held device of any of clauses 1-14, relative to the subject's head, to reposition at least some of the plurality of electrodes on the scalp.

Clause 38. The method according to any of clauses 34-37, wherein the hand-held device comprises or is in communication with a user interface and the placing and/or the adjusting are according to instructions provided by the user interface.

Clause 39. The method according to any of clauses 34-38, wherein the recording comprises setting the electrodes in a bipolar montage or in a weighted average montage.

Clause 40. The method according to clause 39, wherein in the bipolar montage, one of the plurality of electrodes is used as a reference and ground and in the weighted average montage one of the plurality of electrodes is used as common average.

Clause 41. The method according to any of clauses 34-40, comprising repeating the contacting and recording at a plurality of different positions relative to the subject's scalp.

Clause 42. The method according to any of clauses 34-41, wherein the indication related to a condition of the subject is generated by determining patterns in the recorded EEG signals using feature extraction techniques and comparing the patterns to pre-established EEG patterns known to be associated with physiological and/or pathophysiological conditions.

Clause 43. The method according to any of clauses 34-42, wherein the indication related to a condition of the subject is from the group of: a cerebrovascular disease, central nervous system tumor, encephalitis, head trauma, epilepsy, encephalopathy, headache, back pain, spinal cord pathology, multiple sclerosis, other myelopathy, limb weakness/numbness, visual disturbance, unilateral hearing loss.

Clause 44. A method for stimulating a subject and detecting a response to the stimulation using a hand-held device comprising a plurality of electrodes, the method comprising: applying an electrical stimulation to an affected limb; and using the hand-held device, recording a response to the stimulation via the plurality of electrodes.

Clause 45. The method according to clause 44, wherein the electrodes comprise EEG electrodes.

Clause 46. The method according to either of clauses 44 or 45, wherein the response comprises an evoked potential of the nervous system.

Clause 47. The method according to any of clauses 44-46, wherein the applying is via a stimulating device comprising at least an anode, cathode and grounding elements.

Clause 48. The method according to any of clauses 44-47, wherein the recording comprises positioning the hand-held device on the subject's head such that the electrodes are in contact with the scalp.

Clause 49. The method according to any of clauses 44-48, wherein the recording comprises positioning the hand-held device on any anatomical area such that the electrodes contact the skin.

Clause 50. The method according to any of clauses 44-49, wherein the applying an electrical stimulation is to a subject's upper limb, lower limb, or both upper limb and lower limb.

Clause 51. The method according to any of clauses 44-50, wherein the recording is performed at one or more locations along a neural path leading to the stimulated anatomical area.

Clause 52. A hand-held device for recording of electrophysiological data from a subject, comprising:
a flexible substrate, the substrate forming a substantially fixed 3-dimensional shape;
at least three electrodes, each electrode mounted on an external surface of the flexible substrate; and
circuitry for activating the electrodes to record electrophysiology related signals from a subject and for communicating the recorded signals to a remote device.

Clause 53. The device according to clause 52, wherein the electrophysiology data comprises at least one of: Electroencephalography (EEG), Electrocardiogram (ECG) data; Nerve Conduction Velocity (NCV) data; Visual Evoked Potentials (VEP); Brainstem Auditory Evoked Potentials (BAEP); Somatosensory Evoked Potentials (SSEP).

Clause 54. A hand-held device for EEG recording of a subject, comprising:
a flexible substrate having a plurality of electrodes mounted on an external surface of the substrate, the electrodes shaped for contacting the subject's scalp for recording EEG signals; and
circuitry configured to acquire and process signals recorded from a subject by the electrodes and for communicating the recorded signals to a remote device.

Clause 55. The device according to any of clauses 1-16 wherein the subject is a human patient.

Clause 56. The system according to any of clauses 17-32 wherein the subject is a human patient.

Clause 57. The kit according to clause 33 wherein the subject is a human patient.

Clause 58. The method according to any of clauses 34-43 wherein the subject is a human patient.

Clause 59. The method according to any of clauses 44-51 wherein the subject is a human patient.

Clause 60. The device according to either of clauses 52 or 53 wherein the subject is a human patient.

Clause 61. The device according to clause 54 wherein the subject is a human patient.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A hand-held device for electrophysiology data recording from a subject, comprising:
a flexible substrate having a plurality of electrodes mounted onto an external surface of the substrate; and
circuitry configured to acquire and process electrophysiology data signals recorded from the subject by the electrodes and for communicating the recorded electrophysiology data signals to a remote device.

2. The device according to claim 1, wherein the flexible substrate comprises an electrically insulating material.

3. The device according to claim 2, wherein the flexible substrate comprises a reverse surface lined with an electrically insulating lining.

4. The device according to claim 1, wherein the circuitry includes an analog to digital converter, an amplifier, and a communication module.

5. The device according to claim 1, wherein each of the two or more electrodes are at a fixed distance relative to each other electrode on the external surface of the substrate.

6. The device according to claim 1, wherein the electrodes are dry electrodes, each electrode comprising a plurality of pins long enough for contacting the subject's scalp through hair.

7. The device according to claim 1, wherein the circuitry comprises a controller programmed to control recording by the electrodes based on input received from a remote device.

8. The device according to claim 1, comprising one or more optical markers positioned on the external surface, the optical markers detectable by the remote device.

9. The device according to claim 1, further comprising one or more near-infrared spectroscopy sensors on the external surface of the substrate.

10. The device according to claim 1, wherein the electrophysiological data is selected from any of: EEG, ECG, NCV, BAEP, and SSEP.

11. The device according to claim 1, wherein the electrodes are arranged in an x×y matrix, wherein x and y are, independently, integers from 1 to 10.

12. A system comprising:
a device according to claim 1, and
a remote device including a user interface, the user interface configured for receiving input data and for generating a subject condition related indication based on the input data and on the electrophysiological signals recorded by the electrodes.

13. A kit comprising:
a set of two hand-held devices according to claim 1, wherein at least one of the devices further comprises a stimulation module for applying an electrical, visual and/or audible stimulation to the subject.

14. A method for recording an EEG of a subject using the hand-held device of claim 1 comprising a plurality of electrodes, comprising:
contacting the plurality of electrodes with the subject's scalp, resulting in electrically conductive contact between the subject's scalp and at least two electrodes; and
recording EEG signals.

15. The method according to claim 14, further comprising obtaining an automatically generated indication related to a condition of the subject, the indication at least partially based on the recorded EEG signals.

16. The method according to claim 14, comprising adjusting a position of the hand-held device, relative to the subject's head, to reposition at least some of the plurality of electrodes on the scalp.

17. The method according to claim 14, wherein the hand-held device comprises or is in communication with a user interface and the placing and/or the adjusting are according to instructions provided by the user interface.

18. The method according to claim 14, wherein the recording comprises setting the electrodes in a bipolar montage or in a weighted average montage.

19. The method according to claim 18, wherein in the bipolar montage, one of the plurality of electrodes is used as a reference and ground and in the weighted average montage one of the plurality of electrodes is used as common average.

20. The method according to claim 14, wherein the indication related to a condition of the subject is generated by determining patterns in the recorded EEG signals using feature extraction techniques and comparing the patterns to pre-established EEG patterns known to be associated with physiological and/or pathophysiological conditions.

21. A method for stimulating a subject and detecting a response to the stimulation using the hand-held device of claim 1 comprising a plurality of electrodes, the method comprising:
applying an electrical stimulation to an affected limb; and
using the hand-held device, recording a response to the stimulation via the plurality of electrodes.

22. A hand-held device for recording of electrophysiological data from a subject, comprising:
a flexible substrate, the substrate forming a substantially fixed 3-dimensional shape;
at least three electrodes, each electrode mounted on an external surface of the flexible substrate; and
circuitry for activating the electrodes to record electrophysiology related signals from a subject and for communicating the recorded signals to a remote device.
